(19) United States
(12) Patent Application Publication
(10) Pub. No.: US 2026/0007352 A1
(43) Pub. Date: Jan. 8, 2026

(54) AI-BASED ATRIAL FIBRILLATION WARNING SYSTEM USING DYNAMIC ELECTROCARDIOGRAM

(71) Applicant: PLA General Hospital of Northern Theater Command, Shenyang City (CN)

(72) Inventors: Huishan WANG, Shenyang City (CN); Yuji ZHANG, Shenyang City (CN); Yi WU, Shenyang City (CN); Shusheng XU, Shenyang City (CN); Yueyang WANG, Shenyang City (CN); Yongbo SONG, Shenyang City (CN)

(21) Appl. No.: 18/891,331

(22) Filed: Sep. 20, 2024

(30) Foreign Application Priority Data
Jul. 4, 2024 (CN) 202410896584.9

(51) Int. Cl. A61B 5/361; A61B 5/00; A61B 5/332; G06N 3/0455; G06N 3/08; G16H 50/30

(52) U.S. Cl. CPC A61B 5/361; A61B 5/332; A61B 5/7203; A61B 5/7221; A61B 5/746; G06N 3/0455; G06N 3/08; G16H 50/30; A61B 2505/05

(57) ABSTRACT

This application relates to the technical field of medical equipment and health monitoring systems, and provides an artificial intelligence (AI)-based atrial fibrillation warning system using a dynamic electrocardiogram. The system includes: a data acquisition module, a data processing module, an AI analysis module, an alarm mechanism module, and a clinical application module. The data acquisition module is configured to perform continuous electrocardiogram monitoring using a portable dynamic electrocardiogram recorder; the data processing module is configured to preprocess electrocardiogram data; the AI analysis module is configured to train and analyze the preprocessed electrocardiogram data using a deep learning model; the alarm mechanism module is configured to issue an alarm when multiple predictions indicate a risk of atrial fibrillation; and the clinical application module is configured to provide real-time warning assessments that are combined with traditional clinical evaluations.

### CROSS REFERENCE TO RELATED APPLICATION

This patent application claims the benefit and priority of Chinese Patent Application No. 2024108965849, filed with the China National Intellectual Property Administration on Jul. 4, 2024, the disclosure of which is incorporated by reference herein in its entirety as part of the present application.

### TECHNICAL FIELD

The present disclosure relates to the technical field of medical equipment and health monitoring systems, and in particular, to an artificial intelligence (AI)-based atrial fibrillation warning system using a dynamic electrocardiogram.

### BACKGROUND

The main function of the heart is to provide the power for blood flow, circulating blood to various parts of the body. The complete cycle of heart contraction and relaxation is called a heartbeat cycle, which includes atrial contraction, atrial relaxation, ventricular contraction, and ventricular relaxation. Normal heartbeats have a fixed rhythm and sequence. Atrial fibrillation, also known as AFib, is one of the most common types of cardiac arrhythmias. It is characterized by an irregular atrial rhythm caused by multiple small re-entry circuits initiated by the atria. During AFib, the atria are excited at a high rate. The heart rate is not only significantly faster compared to normal individuals but also irregular, and the atria lose their effective contraction function. The occurrence of AFib is associated with age and underlying disease types, with hypertension being the most common cardiovascular disease that can lead to AFib. Patients with concomitant AFib have a significantly increased risk of thromboembolic complications.

Atrial fibrillation is a common arrhythmia, especially more prevalent after cardiac surgery. Post-cardiac surgery significantly increases the risk of stroke and medical costs in patients. Existing technologies lack early warning methods for post-cardiac surgery AFib. Conventional electrocardiogram monitoring systems fail to provide sufficient warning time before AFib occurs, thus failing to effectively prevent its onset and reduce related complications. The purpose of the present disclosure is to provide an AI-based monitoring system using a dynamic electrocardiogram, which can predict the occurrence of AFib in advance. The system uses AI technology for analysis, enabling timely intervention by medical professionals to improve short-term and long-term health outcomes of patients.

### SUMMARY

An objective of the present disclosure is to provide an AI-based atrial fibrillation warning system using a dynamic electrocardiogram, to solve the issues mentioned in the background technology.

To achieve the above objective, the present disclosure provides the following technical solutions: an AI-based atrial fibrillation warning system using a dynamic electrocardiogram, including:
a data acquisition module configured to perform continuous electrocardiogram monitoring using a portable dynamic electrocardiogram recorder;
a data processing module configured to preprocess electrocardiogram data;
an AI analysis module configured to train and analyze the preprocessed electrocardiogram data using a deep learning model;
an alarm mechanism module configured to issue an alarm when multiple predictions indicate a risk of atrial fibrillation; and
a clinical application module configured to provide real-time warning assessments that are combined with traditional clinical evaluations.

Preferably, the data acquisition module requires patients without a history of atrial fibrillation to wear portable dynamic electrocardiogram recorders for seven days before and after cardiac surgery, to continuously monitor cardiac activity and generate detailed electrocardiogram data.

Preferably, in the data acquisition module, all the patients undergo 7-day dynamic electrocardiogram monitoring to ensure that the generated electrocardiogram data covers critical periods before and after the surgery; advanced signal processing algorithms are used to denoise and filter the electrocardiogram data, eliminating motion artifacts and electrode noise.

Preferably, the data processing module is connected to the data acquisition module; in the data processing module, the collected electrocardiogram data undergoes preprocessing, including noise removal and signal interference elimination, and the electrocardiogram data is segmented to ensure independence and representativeness of each data segment.

Preferably, during segmentation of the electrocardiogram data, the continuous electrocardiogram data is divided into independent segments, where each segment contains a plurality of heartbeat signals to enable the deep learning model to identify electrocardiogram features in different states, and each segment is labeled to indicate whether an atrial fibrillation event is present, thereby forming a training set and a validation set.

Preferably, the data processing module is connected to the AI analysis module; the artificial intelligence analysis module optimizes sensitivity and accuracy using a receiver operating characteristic curve pattern and an F1 score pattern, respectively; during a training phase of the AI analysis module, a large-scale dataset is utilized for training the deep learning model, ensuring that the deep learning model possesses high sensitivity and specificity.

Preferably, during model training in the training phase, an AI model combining a one-dimensional convolutional neural network with a Transformer network is constructed; the AI model is trained using the electrocardiogram data, and balanced sampling techniques are employed to address an issue of imbalanced positive and negative samples, ensuring a balanced ratio of positive and negative samples during training; and a stochastic gradient descent optimization algorithm is used during training to continuously adjust model parameters and improve model prediction accuracy.

Preferably, the AI analysis module is connected to the alarm mechanism module; in the alarm mechanism module, the system performs warning assessments every five minutes, providing real-time and reliable warning information in conjunction with the traditional clinical evaluations, and determines whether to issue an alarm based on prediction results from three time intervals; when the prediction results from the three time intervals all indicate a high risk, the system issues an alarm, alerting medical personnel of imminent atrial fibrillation.

Preferably, the alarm mechanism employs a 10-minute integration mode, issuing an alarm only when predictions at 10, 20, and 30-minute intervals all indicate a risk of atrial fibrillation.

Preferably, in the clinical application module, the system continuously monitors and evaluates the electrocardiogram data of the patients in real time, providing reliable warning information in conjunction with the traditional clinical evaluations; medical personnel make timely interventions and adjust medication treatment plans based on the warning information.

Compared with the prior art, the present disclosure has the following beneficial effects:

1. In the present disclosure, 7-day continuous electrocardiogram monitoring of patients is conducted using a portable dynamic electrocardiogram recorder, to collect electrocardiogram data. The data undergoes preprocessing, including denoising and segmentation, to ensure data accuracy and usability. Model training is performed, where deep learning and convolutional neural network models are utilized in conjunction with a Transformer network to train and analyze the electrocardiogram data. The model addresses the issue of data imbalance using balanced sampling techniques to ensure the equilibrium of positive and negative samples. The model predicts imminent atrial fibrillation through a prediction algorithm, including a receiver operating characteristic curve pattern and an F1 score pattern, to optimize sensitivity and accuracy. The system performs multiple predictions at 10, 20, and 30-minute intervals, and issues an alarm when results from all the intervals indicate a risk of atrial fibrillation. Through the clinical application module, the system conducts warning assessments every five minutes in practical applications, providing real-time and reliable warning information in conjunction with traditional clinical evaluations. Analysis is conducted based on the AI technology, allowing medical personnel to intervene timely, thereby improving both short-term and long-term health outcomes for patients.

2. The present disclosure significantly enhances the early warning capability for atrial fibrillation through innovative dynamic electrocardiogram monitoring and AI analysis technology, demonstrating high clinical application value and market potential. The system not only provides timely warning information to medical personnel but also improves patient prognosis through effective intervention measures, thereby reducing related complications and medical costs.

### BRIEF DESCRIPTION OF THE DRAWINGS

FIG. 1 is a system architectural diagram of an AI-based atrial fibrillation warning system using a dynamic electrocardiogram according to a preferred embodiment of the present disclosure;

FIG. 2 is a system architectural diagram of model training according to the present disclosure; and

FIG. 3 is a system architectural diagram of a prediction algorithm according to the present disclosure.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All the other embodiments derived by those of ordinary skill in the art based on the embodiments of the present disclosure without creative efforts shall fall within the protection scope of the present disclosure.

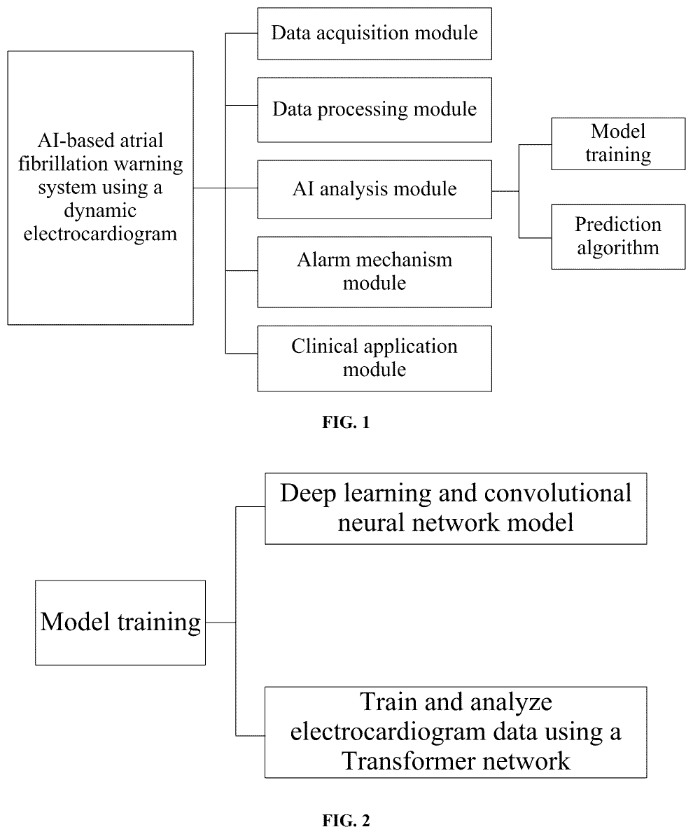

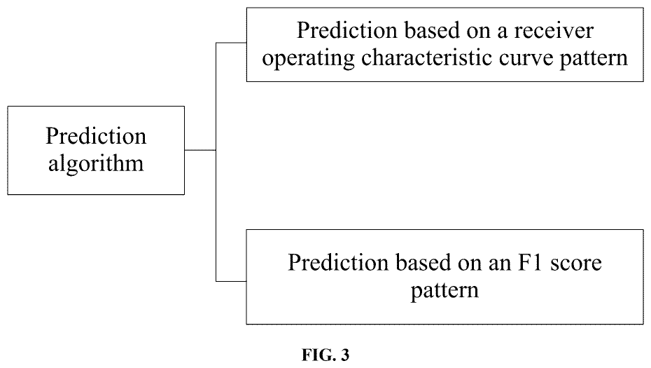

Referring to FIG. 1 to FIG. 3, an AI-based atrial fibrillation warning system using a dynamic electrocardiogram includes:
a data acquisition module configured to perform continuous electrocardiogram monitoring using a portable dynamic electrocardiogram recorder;
a data processing module configured to preprocess electrocardiogram data;
an AI analysis module configured to train and analyze the preprocessed electrocardiogram data using a deep learning model;
an alarm mechanism module configured to issue an alarm when multiple predictions indicate a risk of atrial fibrillation; and
a clinical application module configured to provide real-time warning assessments that are combined with traditional clinical evaluations.

Data collection: Patients without a history of atrial fibrillation wear portable dynamic electrocardiogram recorders for seven days before and after cardiac surgery. The recorder continuously monitors cardiac activity, generating detailed electrocardiogram data. It is essential to ensure that the dynamic electrocardiogram recorder is functioning correctly, and that accessories such as electrodes and lead wires are intact to guarantee the stability and accuracy of data collection. The patients will be informed about the purpose, process, and precautions related to data collection, ensuring they understand and actively cooperate. Before the examination, the patients should avoid vigorous exercise, the consumption of stimulating foods, and medications to ensure the accuracy of data collection. After the recording period, the data from the dynamic electrocardiogram recorder will be downloaded to a computer or storage device to ensure data integrity and accuracy. During this process, data transmission and storage should comply with relevant data security and privacy protection standards. Before the data is submitted to an AI analysis system, preliminary cleaning and organization of the data can be performed to remove noise and outliers, thereby improving the accuracy and efficiency of subsequent analyses.

Data processing: The collected electrocardiogram data undergoes preprocessing, including noise removal and signal interference elimination. The electrocardiogram data is segmented to ensure the independence and representativeness of each data segment. This process includes data slicing: preprocessing the to-be-segmented electrocardiogram data to obtain a plurality of data slices, where each slice contains a plurality of data sampling points that are consecutive in the time dimension.

Model prediction: Each data slice is input into a pre-constructed and trained first segmentation model and second segmentation model to obtain a first segmentation result and a second segmentation result for each data sampling point within each data slice.

Result integration: Based on the first segmentation result and second segmentation result of each data sampling point, overlapping data sampling points between adjacent data slices are filtered and integrated according to a preset rule, to obtain a final electrocardiogram data segmentation result.

Model training and validation: In a training phase, a large-scale dataset is used to train the deep learning model, ensuring high sensitivity and specificity of the model. Data from 545 cardiac surgery patients and 896 non-cardiac surgery patients is used for model training, while data from 249 cardiac surgery patients and 470 non-cardiac surgery patients is used for model validation.

Testing phase: The model undergoes rigorous testing, where data from 308 cardiac surgery patients and 413 non-cardiac surgery patients is used to evaluate model performance.

Clinical application testing: The model is tested in a real clinical environment, using data from 210 cardiac surgery patients and 474 non-cardiac surgery patients to verify practical application effectiveness of the model.

AI analysis module: A one-dimensional convolutional layer is used to process 1,800 heartbeat intervals to extract local features from the electrocardiogram data. A Transformer network is used to identify long-range dependencies and complex patterns in time series data based on an attention mechanism, thereby outputting predicted probabilities of atrial fibrillation and issuing warnings based on the predicted probabilities.

Alarm mechanism: The system conducts warning assessments every five minutes, determining whether to issue an alarm based on prediction results from three time intervals (10, 20, and 30 minutes). The system issues a warning alarm when prediction results of the three time intervals all indicate high risk, alerting medical personnel of imminent atrial fibrillation.

Clinical application: The system continuously monitors and evaluates the electrocardiogram data of the patients in real time, providing reliable warning information in conjunction with the traditional clinical evaluations; medical personnel make timely interventions based on the warning information, such as adjusting medication treatment plans, thereby reducing the occurrence of cardiac surgery.

The AI model of the present disclosure demonstrates a sensitivity of over 93% and a specificity of over 97% in predicting atrial fibrillation, significantly enhancing the early warning capability. Real-time warning is achieved: the system can provide early warnings of atrial fibrillation within 10 to 30 minutes, providing sufficient time for preventive interventions. Medical costs are reduced: early warnings and interventions decrease the occurrence of complications related to cardiac surgery, thereby lowering medical costs. Patient prognosis is improved: Timely interventions can enhance both short-term and long-term health outcomes for patients, reducing the rates of stroke and mortality.

Detailed implementation steps are as follows:

Data acquisition and preprocessing: All patients undergo 7-day dynamic electrocardiogram monitoring to ensure that data covers critical periods before and after surgery; advanced signal processing algorithms are used to denoise and filter the data, eliminating motion artifacts and electrode noise.

Data segmentation and labeling: The continuous electrocardiogram data is divided into independent segments, where each segment contains 1,800 heartbeat signals to enable the model to identify electrocardiogram features in different states, and each segment is labeled to indicate whether an atrial fibrillation event is present, thereby forming a training set and a validation set.

Model training: An AI model combining a one-dimensional convolutional neural network (CNN) with a Transformer network is constructed; the AI model is trained using the electrocardiogram data, and balanced sampling techniques are employed to address an issue of imbalanced positive and negative samples, ensuring a balanced ratio of positive and negative samples during training; and a stochastic gradient descent (SGD) optimization algorithm is used during training to continuously adjust model parameters and improve model prediction accuracy.

Model validation and testing: Model performance is evaluated using the validation set, including metrics such as sensitivity, specificity, accuracy, and precision. The generalization ability of the model is further validated on a test set to ensure the stability and reliability of the model across different datasets.

Alarm mechanism setup: The system performs predictive evaluations every 5 minutes, and assesses the risk of atrial fibrillation based on prediction results from the past 10, 20, and 30 minutes. Based on a 10-minute integration mode, the system issues an alarm when prediction results of all time intervals indicate high risk.

Clinical application and feedback: The system is applied in a real clinical environment to monitor electrocardiogram data of patients in real time. Interventions are made based on AI warning information, and clinical feedback information is collected to continuously optimize and enhance the system, thereby improving warning accuracy and practicality.

Clinical practical application: Following the clinical application, the specific implementation is as follows:

Patient screening: Adult patients undergoing cardiac surgery receive 7 days of dynamic electrocardiogram monitoring before and after surgery. Patients with a history of atrial fibrillation, those aged 80 and above, and emergency surgery cases are excluded. Monitoring and intervention: Patients wear portable dynamic electrocardiogram recorders for continuous monitoring of cardiac activity. The system analyzes electrocardiogram data every 5 minutes and predicts the occurrence of atrial fibrillation based on an AI algorithm. When atrial fibrillation risk is predicted, the system alerts the medical team. In clinical practice: The medical team, guided by system alerts and traditional clinical evaluations, determines whether to take preventive intervention measures such as adjusting medication dosages or enhancing monitoring, to reduce the risk of atrial fibrillation occurrence. Data analysis and feedback: Clinical data and feedback information are collected to evaluate the effectiveness of the system in real-world applications. The algorithm is optimized continuously, to improve prediction accuracy and alarm reliability.

The working principle of the present disclosure is as follows: Data acquisition and preprocessing: All patients undergo 7-day dynamic electrocardiogram monitoring to ensure that data covers critical periods before and after surgery; advanced signal processing algorithms are used to denoise and filter the data, eliminating motion artifacts and electrode noise. Data segmentation and labeling: The continuous electrocardiogram data is divided into independent segments, where each segment contains 1,800 heartbeat signals to enable the model to identify electrocardiogram features in different states, and each segment is labeled to indicate whether an atrial fibrillation event is present, thereby forming a training set and a validation set. Model training: An AI model combining a one-dimensional convolutional neural network (CNN) with a Transformer network is constructed; the AI model is trained using the electrocardiogram data, and balanced sampling techniques are employed to address an issue of imbalanced positive and negative samples, ensuring a balanced ratio of positive and negative samples during training; and a stochastic gradient descent (SGD) optimization algorithm is used during training to continuously adjust model parameters and improve model prediction accuracy. Model validation and testing: Model performance is evaluated using the validation set, including metrics such as sensitivity, specificity, accuracy, and precision; the generalization ability of the model is further validated on a test set to ensure the stability and reliability of the model across different datasets. Alarm mechanism setup: The system performs predictive evaluations every 5 minutes, and assesses the risk of atrial fibrillation based on prediction results from the past 10, 20, and 30 minutes; based on a 10-minute integration mode, the system issues an alarm when prediction results of all time intervals indicate high risk. Clinical application and feedback: The system is applied in a real clinical environment to monitor electrocardiogram data of patients in real time; interventions are made based on AI warning information, and clinical feedback information is collected to continuously optimize and enhance the system, thereby improving warning accuracy and practicality.

It should be noted that relational terms herein such as first and second are merely used to distinguish one entity or operation from another entity or operation without necessarily requiring or implying any actual such relationship or order between such entities or operations. In addition, terms “include”, “comprise”, or their any other variations are intended to cover a non-exclusive inclusion, such that a process, a method, an article, or a device that includes a series of elements not only includes those elements, but also includes other elements that are not explicitly listed, or also includes inherent elements of the process, the method, the article, or the device. In the case that there are no more restrictions, an element limited by the statement “includes a . . . ” does not exclude the presence of additional identical elements in the process, the method, the article, or the device that includes the element.

Although the embodiments of the present disclosure have been illustrated and described, it should be understood that those of ordinary skill in the art may make various changes, modifications, replacements and variations to the above embodiments without departing from the principle and spirit of the present disclosure, and the scope of the present disclosure is limited by the appended claims and their legal equivalents.

What is claimed is:

1. An artificial intelligence (AI)-based atrial fibrillation warning system using a dynamic electrocardiogram, comprising:
a data acquisition module configured to perform continuous electrocardiogram monitoring using a portable dynamic electrocardiogram recorder;
a data processing module configured to preprocess electrocardiogram data;
an AI analysis module configured to train and analyze the preprocessed electrocardiogram data using a deep learning model;
an alarm mechanism module configured to issue an alarm when multiple predictions indicate a risk of atrial fibrillation; and
a clinical application module configured to provide real-time warning assessments that are combined with traditional clinical evaluations.

2. The AI-based atrial fibrillation warning system using a dynamic electrocardiogram according to claim 1, wherein the data acquisition module requires patients without a history of atrial fibrillation to wear portable dynamic electrocardiogram recorders for seven days before and after cardiac surgery, to continuously monitor cardiac activity and generate detailed electrocardiogram data.

3. The AI-based atrial fibrillation warning system using a dynamic electrocardiogram according to claim 2, wherein in the data acquisition module, all the patients undergo 7-day dynamic electrocardiogram monitoring to ensure that the generated electrocardiogram data covers critical periods before and after the surgery; advanced signal processing algorithms are used to denoise and filter the electrocardiogram data, eliminating motion artifacts and electrode noise.

4. The AI-based atrial fibrillation warning system using a dynamic electrocardiogram according to claim 1, wherein the data processing module is connected to the data acquisition module; in the data processing module, the collected electrocardiogram data undergoes preprocessing, comprising noise removal and signal interference elimination, and the electrocardiogram data is segmented to ensure independence and representativeness of each data segment.

5. The AI-based atrial fibrillation warning system using a dynamic electrocardiogram according to claim 4, wherein during segmentation of the electrocardiogram data, the continuous electrocardiogram data is divided into independent segments, wherein each segment contains a plurality of heartbeat signals to enable the deep learning model to identify electrocardiogram features in different states, and each segment is labeled to indicate whether an atrial fibrillation event is present, thereby forming a training set and a validation set.

6. The AI-based atrial fibrillation warning system using a dynamic electrocardiogram according to claim 1, wherein the data processing module is connected to the AI analysis module; the artificial intelligence analysis module optimizes sensitivity and accuracy using a receiver operating characteristic curve pattern and an F1 score pattern, respectively; during a training phase of the AI analysis module, a large-scale dataset is utilized for training the deep learning model, ensuring that the deep learning model possesses high sensitivity and specificity.

7. The AI-based atrial fibrillation warning system using a dynamic electrocardiogram according to claim 6, wherein during model training in the training phase, an AI model combining a one-dimensional convolutional neural network with a Transformer network is constructed; the AI model is trained using the electrocardiogram data, and balanced sampling techniques are employed to address an issue of imbalanced positive and negative samples, ensuring a balanced ratio of positive and negative samples during training; and a stochastic gradient descent optimization algorithm is used during training to continuously adjust model parameters and improve model prediction accuracy.

8. The AI-based atrial fibrillation warning system using a dynamic electrocardiogram according to claim 1, wherein the AI analysis module is connected to the alarm mechanism module; in the alarm mechanism module, the system performs warning assessments every five minutes, providing real-time and reliable warning information in conjunction with the traditional clinical evaluations, and determines whether to issue an alarm based on prediction results from three time intervals; when the prediction results from the three time intervals all indicate a high risk, the system issues an alarm, alerting medical personnel of imminent atrial fibrillation.

9. The AI-based atrial fibrillation warning system using a dynamic electrocardiogram according to claim 8, wherein that the alarm mechanism employs a 10-minute integration mode, issuing an alarm only when predictions at 10, 20, and 30-minute intervals all indicate a risk of atrial fibrillation.

10. The AI-based atrial fibrillation warning system using a dynamic electrocardiogram according to claim 1, wherein in the clinical application module, the system continuously monitors and evaluates the electrocardiogram data of the patients in real time, providing reliable warning information in conjunction with the traditional clinical evaluations; medical personnel make timely interventions and adjust medication treatment plans based on the warning information.
